(19) United States
(12) Patent Application Publication
(10) Pub. No.: US 2006/0142721 A1
(43) Pub. Date: Jun. 29, 2006

(54) Pouch with wiping capability

(76) Inventor: Cindy Price, Appleton, WI (US)

Correspondence Address: BRINKS HOFER GILSON & LIONE, P.O. BOX 10395, CHICAGO, IL, 60610, US

(21) Appl. No.: 11/025,663

(22) Filed: Dec. 29, 2004

(51) Int. Cl. A61F 13/15

(52) U.S. Cl. 604/385.02

(57) ABSTRACT

An individually wrapped absorbent article includes an absorbent article and a unitary sheet of material configured as a pouch surrounding the absorbent article. The unitary sheet of material includes an interior surface and an exterior surface. At least one of the interior and exterior surfaces has a liquid absorbent capacity greater than about 10 weight percent.

### TECHNICAL FIELD

This invention relates generally to individually wrapped absorbent articles. More particularly, this invention relates to a wrapper material with wiping capability.

### BACKGROUND OF THE INVENTION

Absorbent care articles such as sanitary napkins, panty liners, and other types of catamenial devices are used to absorb menses and other body fluids. These absorbent products are primarily disposable. Since many of these articles are carried in a purse or pocket prior to use, it is advantageous to individually wrap each article to keep it clean and sanitary as well as to increase the discretion of the article. By individually packaging each absorbent article, it can be assured that the article will not become contaminated by the contents of the user's purse, pocket, etc.

Conventionally, the article wrapper or pouch consists of one or more layers of a thin sheet or film of thermoplastic material, such as polyethylene, which is folded around the absorbent article and then sealed by the use of heat and/or pressure, ultrasonics, or an adhesive to form a pouch. The pouch is designed to be opened by breaking or tearing the material at or adjacent a seal in order to subsequently remove the absorbent article or to open the front portion of the pouch while exposing the article to the user. Conventional pouches are also typically designed so that a soiled article may be wrapped up in the opened pouch for later disposal.

However, commercial pouches are traditionally made of hydrophobic, relatively continuous materials which make them unacceptable to use for wiping or absorbing bodily fluids. Consequently, it is desirable to deliver a pouch that has a capacity for absorbing liquids. Such a pouch could ideally be used by the consumers to wipe themselves, as a hygienic package for product delivery, and also as a wrapper that could absorb fluid or particulates that a used article may leak after it is wrapped into the pouch.

### SUMMARY OF THE INVENTION

According to an aspect of the present invention, the individually wrapped absorbent article includes an absorbent article and a unitary sheet of material configured as a pouch surrounding the absorbent article. The unitary sheet of material includes an interior surface and an exterior surface. At least one of the interior and exterior surfaces has a liquid absorbent capacity greater than about 10 weight percent.

According to another aspect of the invention, a method of making the individually wrapped absorbent article includes providing a sheet of material comprising an interior surface and an exterior surface, placing an absorbent article on the interior surface, and configuring the sheet into a pouch surrounding the absorbent article such that the interior surface is on the interior of the pouch. At least one of the interior and exterior surfaces has a liquid absorbent capacity greater than about 10 weight percent.

According to a further aspect of the invention, a method of using the individually wrapped absorbent article includes unwrapping a pouch surrounding an absorbent article, the pouch forming from a unitary sheet of material having an interior surface and an exterior surface, separating the sheet from the absorbent article, wiping a portion of a body surface proximate the area to which the absorbent article is to be applied with the a surface of the sheet, applying the absorbent article, and disposing of the sheet of material. At least one of the interior and exterior surfaces has a liquid absorbent capacity greater than about 10 weight percent.

### BRIEF DESCRIPTION OF THE DRAWINGS

FIG. 1 depicts a perspective view of an individually wrapped absorbent article.

FIG. 2 depicts a sheet used to form the individually wrapped absorbent article of FIG. 1.

FIG. 3 depicts a cross-section perspective view of an absorbent article used to form the individually wrapped absorbent article of FIG. 1.

FIG. 4 depicts a close up front view of FIG. 3.

FIG. 5 depicts another perspective view of FIG. 1 shown in its open state.

### DETAILED DESCRIPTION

A better understanding of the present invention will now be had upon reference to the following detailed description, when read in conjunction with the accompanying drawings, wherein like reference characters refer to like parts throughout the several views of the present invention.

An individually wrapped absorbent article 18 includes an absorbent article 20 and a unitary sheet of material 22 configured as a pouch 24 surrounding the absorbent article 20, as depicted in FIG. 1. The pouch 24 provides a sanitary environment for storing and carrying the absorbent article 20 before it is ready to be used, and may be conveniently unwrapped to release the absorbent article 20 for use. The pouch 24 can also provide discretion and privacy for the user. The sheet 22 may be used for wiping, cleaning and/or drying the skin and/or hair, for disposing the used absorbent article 20, and may contain other additives for specific purposes.

The term “a unitary sheet of material” means a single, discrete layer of material that cannot be separated into two or more component layers. A unitary sheet of material may be made of a single substance, or it may be a composite of two or more substances. A composite unitary sheet of material may have a homogenous composition, or it may have a composition that varies along the width, length and/or thickness of the sheet. The two opposing surfaces of a unitary sheet may be the same or they may be different. For example, a composite sheet in which the composition varies across the thickness may have different surface properties on each opposing surfaces, due to the difference in chemical composition. In another example, a unitary sheet may be subjected to different processing steps on each surface, such as drying, creping or abrading.

The unitary sheet of material 22 comprises an interior surface 26 and an exterior surface 28, as depicted in FIG. 2. At least one of the interior 26 and exterior surfaces 28 has a liquid absorbent capacity greater than about 10 weight percent. The liquid absorbent capacity refers to the capacity of a material to absorb liquid over a period of time and is related to the total amount of liquid held by a material at its point of saturation. Liquid absorbent capacity is determined by measuring the increase in the weight of a material sample resulting from the absorption of a liquid. Liquid absorbent capacity may be expressed, in percent, as the weight of liquid absorbed divided by the initial weight of the sample by the following equation:
Total Absorptive Capacity=[(saturated sample weight−initial sample weight)/initial sample weight]×100

It is noted that the absorbent capacity is calculated with respect to the weight of the entire sheet, even though only one of the surfaces may provide for liquid absorbance. If a sheet has only one surface that provides for liquid absorbance, then application of a liquid to the non-absorbent surface will result in a calculated absorbent capacity less than 10 weight percent due to the prevention of liquid absorbance by this surface. This same sheet, however, when contacted with a liquid on the opposite absorbent surface, will result in a calculated absorbent capacity of greater than about 10 weight percent.

A sheet surface may exhibit a liquid absorbent capacity greater than about 10 weight percent through a variety of mechanisms. For example, the chemical nature of the sheet components at the surface may provide for incorporation of the liquid into the component itself. Examples of such components include absorbent pulp fibers and absorbent or superabsorbent particles. In another example, the chemical nature of the sheet surface may provide a hydrophilic surface that facilitates the transfer of aqueous liquids from an object to the sheet. The term “hydrophilic” means of, relating to, or having a strong affinity for water. The term “hydrophobic” means lacking affinity for water. A hydrophilic surface exhibits a water-in-air contact angle of less than 90° (i.e., 0° to 90°). In yet another example, the physical conformation of the surface may provide for trapping of a liquid on or in the surface. A surface with substantial loft and/or void volume can pick up liquid and/or particulates from an object, even if the surface components do not incorporate the liquid into their structure or are not inherently wetted by the liquid.

The sheet 22 may be made from a high pulp content nonwoven composite fabric. An example of a high pulp content nonwoven composite fabric is the Hydroknit® material, available from Kimberly-Clark Corporation, which includes pulp fibers mixed with a continuous filament nonwoven substrate. The term “nonwoven” means a material having a structure of individual fibers or threads that are interlaid, but not in any identifiable, repeating manner. For example, the pulp fiber may be any low-average fiber length pulp or high average fiber length pulp, or mixture of the same. Examples of low-average fiber length pulps include certain grades of virgin hardwood pulp and low-quality secondary (i.e. recycled) fiber pulp from sources such as newsprint, reclaimed paperboard, and office waste. Examples of high-average fiber length pulps include bleached and unbleached virgin softwood pulps. The continuous filament nonwoven substrate may be a nonwoven web of continuous melt-spun filaments formed by a spunbond process. For example, the filaments may be formed from polyolefins such as polypropylene or polyethylene; polyamides; polyesters; polyurethanes; filaments may also be formed from thermoplastic elastomers, such as A-B and A-B-A′ block copolymers where A and A′ are thermoplastic endblocks and B is an elastomeric midblock; and copolymers of ethylene and at least one vinyl monomer such as vinyl acetates, unsaturated aliphatic monocarboxylic acids, and esters of such monocarboxylic acids.

The pulp and the continuous filament substrate are combined to form one strong and absorbent unitary sheet of material. No glues or binders are used to combine the substances, which makes the material safe and effective when used with additives such as yeast infection treatments, deodorants, fragrance, lubricants, botanical extracts and powders. The pulp provides the softness and the absorbency while the nonwoven provides the strength and toughness. An example of a high pulp content nonwoven composite fabric is described in U.S. Pat. No. 5,284,703, the entire disclosure of which is incorporated herein by reference.

The sheet 22 may also be made from an extensible fabric. In this way, the sheet 22 is made of a material that is extensible while not retractable, which provides better handling for the user, especially in the case of small pouches. For example, the extensible fabric may be a spunbond fabric. The term “spunbond fabric” means small diameter fibers of molecularly oriented polymeric material. In one example, the material may include a nonwoven, wire-weave spunbond polypropylene fabric composed of about 1.6 denier fibers formed into a web having a basis weight of about 0.6 osy. One suitable non-woven material is the Corinth 0.60 osy, 1.6 dpf wireweave, nonwettable Metallocene (EXXON ACHIEVE 2854 PP) spunbond material manufactured by Kimberly-Clark Corporation. In another example, the material may include a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.28% Triton X-102 surfactant. The surfactant can be applied by any conventional means, such as spraying, printing, brush coating or the like. In yet another example, the material may include spunbond fabrics such as a 60% necked, polypropylene spunbond having a basis weight of about 1.2 osy.

The sheet 22 may also be made from a coform material. The term “coform” means composite materials including a mixture or stabilized matrix of thermoplastic filaments and at least one additional material, usually called the second material. Examples of coform materials are described in U.S. Patent Application Publication No. 2003/0211802, filed Dec. 9, 2002, and is assigned to Kimberly-Clark Corporation, the entire disclosure of which is incorporated herein by reference. The sheet 22 may also be made from an airlaid tissue, such as the one described in U.S. Patent Application Publication No. 2004/0192136, filed Mar. 25, 2003, and is assigned to Kimberly-Clark Corporation, the entire disclosure of which is incorporated herein by reference. The sheet 22 may also be made from an uncreped through-air dried (UCTAD) tissue, such as the one described in U.S. Pat. No. 5,607,551, and is assigned to Kimberly-Clark Corporation, the entire disclosure of which is incorporated herein by reference. The sheet may also be made from paper towel material, such as the materials described in U.S. Pat. No. 6,423,180, assigned to Kimberly-Clark Corporation, the entire disclosure of which is incorporated herein by reference. A specific example of paper towel material is VIVA paper towel material, such the materials described in U.S. Pat. Nos. 6,436,234; 6,565,707 and 6,727,004; all of which are assigned to Kimberly-Clark Corporation, the entire disclosures of which are incorporated herein by reference.

The sheet 22 may be flushable, such that the sheet and a used absorbent article can be disposed of by flushing in a toilet. Flushable sheets typically contain a fibrous material and a binder composition that binds the fibrous material into a coherent fibrous substrate. A flushable sheet desirably has adequate wet strength when used for wiping liquid from a surface, but disperses when placed in a large excess of water and/or when subjected to the shear forces that occur when a toilet is flushed. Examples of flushable sheet materials are described in U.S. Pat. Nos. 5,948,710 and 5,952,251, which are incorporated herein by reference. The components of the flushable sheet may become water-dispersible by one or more of a number of triggering mechanisms, including pH change, temperature change, or change in the type and concentration of ions in an aqueous environment. Examples of temperature-sensitive flushable sheets are described, for example, in U.S. Pat. Nos. 5,969,052; 6,585,922; 6,664,333, all of which are incorporated herein by reference. Examples of ion-sensitive flushable sheets are described, for example, in U.S. Pat. Nos. 6,043,317; 6,423,804; 6,429,261; 6,537,663; 6,828,014 and in U.S. Patent Application Publications 2003/0220042; 2004/0058073; and 2004/0063888, all of which are incorporated herein by reference.

The sheet 22 may be an elongated rectangular piece having a first end 34, an opposite second end 36, and generally parallel longitudinal sides 38 and 40 extending between the ends 34 and 36, as depicted in FIG. 2. The interior surface 26 may have a smaller area or may have the same area as the exterior surface 28. In one example, the interior surface 26 may be moist or dry. The interior surface 26 may also have at least one moist region 42 and at least one dry region 44. In another example, the interior surface 26 may have a hydrophilic surface 45 and the exterior surface 28 may have a hydrophobic surface 47. The interior hydrophilic surface 26 may be dry, or it may be moist. A moist surface region contains from 10-500% added liquid relative to the mass of the surface region when dry. The interior surface 26 may also have mixed hydrophilic 45 and hydrophobic regions 47. For a sheet 22 having a moist interior surface 26, the presence of a hydrophobic surface 47 surrounding the moist hydrophilic region 45 can inhibit the loss of moisture from the hydrophilic region 45.

The absorbent article 20 may be a catamenial device such as a sanitary napkin, a panty liner, a labial pad, an incontinence pad, or any other type of absorbent article which can be used to absorb menstrual fluid, urine, body fluid, body exudate, etc. More than one type of absorbent article 20 may be contained in the same pouch 24. For purposes of describing the individually wrapped absorbent article 18, the absorbent article 20 is shown and referred to herein as a sanitary pad or napkin.

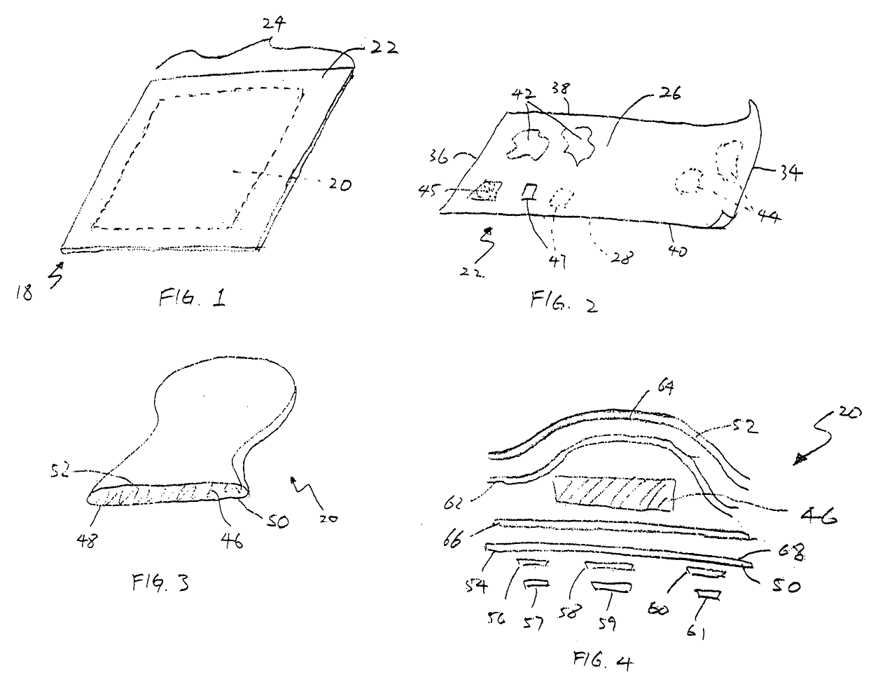

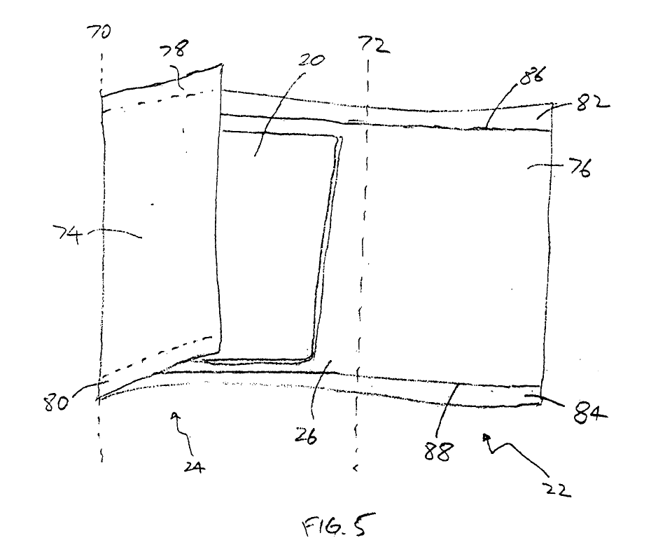

The absorbent article 20 includes an absorbent core 46 and an envelope 48 encasing the absorbent core 46, as depicted in FIG. 3. The absorbent core 46 is designed to absorb body exudates, including menstrual fluid, blood, urine, and other body fluids. The absorbent core 46 can consist of one or more layers of absorbent material. The layers can consist of similar materials or different materials. Examples of the absorbent core 46 include cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, as it is low in cost, relatively easy to form, and has great absorbency. The absorbent core 46 can also be formed from a composite comprised of a hydrophilic material that can be formed from various natural or synthetic fibers, wood pulp fibers, regenerated cellulose or cotton fibers, or a blend of pulp and other fibers. A preferred material is an airlaid tissue.

In one example, the absorbent core 46 also includes a superabsorbent material, which increases the ability of the absorbent core 46 to absorb a large amount of fluid in relation to its own weight. Typical superabsorbents used in absorbent articles such as sanitary napkins, can absorb anywhere from 5 to 60 times their weight in body fluid. The superabsorbent materials can be inserted as particles or in sheet form. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25. Other types of superabsorbent materials known to those skilled in the art can also be used.

The envelope 48 includes a liquid-impervious backsheet or baffle 50, which is disposed below the absorbent core 46, and a liquid-permeable topsheet or cover 52, which is positioned above the absorbent core 46, as depicted in FIGS. 3 and 4. The topsheet 52 is configured to face the user's body and functions to permit liquids to pass through it for retention by the absorbent core 46. The backsheet 50 has a back face 54, which is configured to be releasably secured to the sheet 22 by, for example, three garment adhesive strips or patches 56, 58 and 60, as depicted in FIG. 4. The garment adhesive strips 56, 58 and 60 could be formed from any adhesive and preferably are formed from a pressure-sensitive adhesive of the type commonly used in the art. It should be noted that one or two wide adhesive strips could be used in place of three narrower adhesive strips 56, 58 and 60.

In one example, the absorbent article 20 does not require a separate peel strip or liner and/or pouch because it is provided with the sheet 22 which serves as the pouch 24. In another example, the absorbent article contains peel strips 57, 59 and 61, which correspond to adhesive strips 56, 58 and 60, respectively. For example, silicon-coated papers may be used as peel strips. It should be note that one or two wide peel strips could be used in place of three narrower peel strips 57, 59 and 61. Once the sheet 22 or the peel strips 57, 59 and 61 are removed from the absorbent article 20, the garment adhesive strips 56, 58 and 60 remain with the absorbent article 20 and function to attach and hold the absorbent article 20 in position on the inner surface of the user's undergarment. The topsheet 52 is secured to the backsheet 50 by a construction adhesive 62 located on an inner surface 64 of the topsheet 52 and a bodyside layer of construction adhesive 66 on an inner or front face 68 of the backsheet 50. This construction adhesive 66 could be eliminated in favor of a thermal bond if desired or by other bonding techniques known to those skilled in the art, such as ultrasonics.

Referring to FIG. 5, the sheet 22 can be folded around the absorbent article 20 such that the pouch 24 is formed around the absorbent article 20. The absorbent article 20 can be placed on the interior surface 26 of the sheet 22, and the sheet 22 may then be folded along a pair of fold lines 70 and 72 to form a tri-fold configuration. It should be noted that the sheet 22 can also be bi-folded, flat or rolled. It should also be noted that the absorbent article 20 can also be tri-folded, bi-folded, flat or rolled when it is placed on the sheet 22. In the example of a sheet 22 that is tri-folded, the sheet 22 is folded at a first axis 70 and then at a second axis 72 to define the flaps 74 and 76. The sides 78 and 80 of the flap 74 and the sides 82 and 84 of the flap 76 are then bonded or sealed together. These seals or bonds can be formed by heat and/or pressure, adhesive, ultrasonic bonding, or other types of bonding techniques know to those skilled in the art. Preferably, an ultrasonic or thermal bond is used because an adhesive bond may leave adhesive residue that may stick to the user's body or pubic hair when wiping.

The seals can be made to be a “permanent” seal, which means that the sheet 22 adjacent to the seal will tear or break before the sealed layers separate. The seals may be “frangible” seals, which mean that the sealed layers will separate or pull apart. In one example, the seal between the flap sides 78 and 80 and the flap sides 82 and 84 is a frangible seal formed by simultaneously sealing the flap sides 78 and 80 and flap sides 82 and 84 in a single sealing operation with a heated/pressure embossing roll. It has been found that, compared to sealing purely film sides, a greater variance in the sealing temperature may be used. For example, the temperature of the embossing roll may be maintained between about 245° F. and about 285° F., but can be adjusted depending on machine speeds and operation conditions to achieve the desire result. In another example, the seal between the flap sides 78 and 80 and the flap sides 82 and 84 is a permanent seal formed by simultaneously sealing the flap sides 78 and 80 and flap sides 82 and 84. In this example, the flap sides 82 and 84 each defines perforation lines 86 and 88, respectively, that allow the flap 76 to tear along the perforation lines 86 and 88 and expose the absorbent article 20 when opening the pouch 24.

An individually wrapped article can be formed by a variety of methods. For example, the sheet 22 may be partially formed into a pouch 24, the absorbent article 20 may be placed in the partially formed pouch 24, and then the sheet 22 may be sealed to complete the pouch 24. Various pouch configurations are known and used in the art for individually wrapped absorbent articles, and any such configuration may be used. Wrapping an absorbent article using a wrapper material is described in U.S. Pat. No. 4,556,146 entitled “Individually Packaged Disposable Absorbent Article,” which issued to Swanson et al. on Dec. 3, 1985, and is incorporated herein by reference. In another example, absorbent article 20 is placed in the sheet 22, and the sheet 22 is folded into pouch 24. This example is illustrated in FIG. 1.

The pouch 24 can provide a sanitary environment because the absorbent article 20 is sealed along the sides 38 and 40 by the exterior surface 28 of the sheet 22. Thus, the individually wrapped absorbent article 18 can be hygienically stored in the user's purse or pocket until use. Moreover, if the interior surface 26 is moist, the interior surface 26 will not dry out before use because it is sealed or the absorbent article 20 is configured such that the absorbent core 46 is not in direct contact with the pouch 24, and thus the moisture will not be transferred to the absorbent article 20. In one example, the backsheet 50, the adhesive strips 56, 58 and 60, or the peel strips 57, 59 and 61 are used to form a compartment to retain the moist environment prior to use, and thus moisture will not migrate to dry areas or dry out. In another example, the absorbent article 20 can be tri-folded.

In addition to providing a sanitary environment for the absorbent article 20, the sheet 22 may allow the absorbent article 20 to be carried and used discretely. The sheet 22 may contain high loads of pigment, TiO₂ and/or high basis weight materials to increase the opaqueness of the pouch 24. This opacity can prohibit others from seeing through the individually wrapped absorbent article 18, and can give the user an enhanced sense of privacy and discretion. The sheet 22 may also give the pouch 24 a soft and cloth-like tactile feel and to dampen and reduce noise associated with storing, carrying and opening the pouch 24.

The pouch 24 may be quickly and readily opened and unwrapped simply by pulling the flap 76 away from the flap 74, against the relatively small resistance of the frangible adhesive patch in one example. The side flaps 82 and 84 can be unfolded against the relatively slight resistance of the frangible adhesive strip. The pouch 24 may also be opened and unwrapped by pulling the flap 76 away from the flap 74 by tearing along the perforation lines 86 and 88 in another example. The absorbent article 20 can then be peeled away from the wrapper material 22 simply by grasping one end of the absorbent article 20 and pulling it longitudinally with respect to the sheet 22. The absorbent article 20 is now ready for attachment to an undergarment. The individually wrapped absorbent article 18 may optionally include visual cues or characteristics to assist the opening of the pouch 24. The term “visual characteristic” means a feature or characteristic that is discernible by sight during the normal use of the individually wrapped absorbent article 18, and includes for example and without limitation color, shape, embossing, patterns (e.g. by printing), sealing patterns, tabs, slits, etc.

The interior surface 26 of the unfolded sheet 22 has a liquid absorbent characteristic that is useful for wiping and/or cleaning. For example, the interior surface 26 may be used as a wipe to absorb, trap and pick up bodily fluids and particulates or to clean the user's body before or after the absorbent article 20 is used. In the example where the exterior surface 28 is also hydrophilic, the exterior surface 28 may also be used for wiping and/or cleaning. Moreover, the interior surface 26 may contain additives like treatments for yeast infections, deodorants, fragrances and lubricants. For example, older women who suffer from increased vaginal dryness could benefit from a wipe having a vaginal lubricant. Furthermore, the sheet 22 can contain a powder, such as talc, baking soda, carbon based for odor control, below the interior surface 26 that would migrate to the top when the user was ready to change and wipe.

After use, the absorbent article 20 can be conveniently and discretely disposed of by simply wrapping it in the sheet 22. This wrapping and subsequent disposal are facilitated by the fact that the sheet 22 is substantially larger than the absorbent article 20 in both the longitudinal and lateral directions. Thus, there is no need to force a used absorbent article in a separate formed pouch during disposal. Moreover, the hydrophilic properties of the sheet 22 are configured to absorb some of the fluids and/or particulates that can be leaked by the absorbent article 20 while it is being positioned for disposal. Thus, there is no need to wrap toilet paper around the used absorbent article 20. In one example, if the sheet 22 and the used absorbent article 20 are flushable, then they can be disposed of in a toilet, either together or separately. In another example, the exterior surface 28 of the sheet material 22 may be hydrophobic and can be used as a hygienic package for transport of the used absorbent article 20. Moreover, the used absorbent article 20 in the sheet 22 may be tacked to hold the sheet 22 closed. For tacking, a tab, adhesive spot, or other means can be used on the exterior surface 28 of the sheet 22. This improves the hygiene of the user handling the disposal.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

What is claimed is:

1. An individually wrapped absorbent article, comprising:
an absorbent article; and
a unitary sheet of material configured as a pouch surrounding the absorbent article, the unitary sheet of material comprising an interior surface and an exterior surface;
wherein at least one of the interior and exterior surfaces has a liquid absorbent capacity greater than about 10 weight percent.

2. The individually wrapped absorbent article of claim 1, wherein the sheet comprises a high pulp content nonwoven composite fabric.

3. The individually wrapped absorbent article of claim 1, wherein the sheet comprises an extensible fabric.

4. The individually wrapped absorbent article of claim 1, wherein the sheet comprises at least one material selected from the group consisting of a coform material, an airlaid tissue, a UCTAD tissue, and a paper towel.

5. The individually wrapped absorbent article of claim 1, wherein the sheet is flushable.

6. The individually wrapped absorbent article of claim 1, wherein the interior surface is dry.

7. The individually wrapped absorbent article of claim 1, wherein the interior surface is moist.

8. The individually wrapped absorbent article of claim 1, wherein the interior surface comprises at least one dry region and at least one moist region.

9. The individually wrapped absorbent article of claim 1, wherein the sheet comprises at least one frangible seal.

10. The individually wrapped absorbent article of claim 1, wherein the sheet has a tri-fold configuration.

11. The individually wrapped absorbent article of claim 1, wherein the sheet comprises a flap defined by a pair of perforation lines.

12. The individually wrapped absorbent article of claim 1, wherein the sheet further comprises at least one additive selected from the group consisting of a yeast infection treatment, a deodorant, a fragrance, a lubricant, a botanical extract, and a powder.

13. The individually wrapped absorbent article of claim 1, wherein at least one of the interior surface and the exterior surface is hydrophilic.

14. The individually wrapped absorbent article of claim 13, wherein the interior surface is hydrophilic and the exterior surface is hydrophobic.

15. The individually wrapped absorbent article of claim 1, wherein the sheet is flushable.

16. A method of making an individually wrapped absorbent article, comprising:
providing a sheet of material comprising an interior surface and an exterior surface;
placing an absorbent article on the interior surface; and
configuring the sheet into a pouch surrounding the absorbent article such that the interior surface is on the interior of the pouch;
wherein at least one of the interior and exterior surfaces has a liquid absorbent capacity greater than about 10 weight percent.

17. The method of claim 16, wherein the configuring further comprises forming the sheet into a partial pouch prior to placing the absorbent article, and forming the partial pouch into a completed pouch after placing the absorbent article.

18. The method of claim 16, wherein the pouch comprises a high pulp content nonwoven composite fabric.

19. The method of claim 16, wherein the sheet comprises at least one material selected from the group consisting of a coform material, an airlaid tissue, a UCTAD tissue, and a paper towel.

20. The method of claim 16, further comprising sealing at least one edge of the pouch.

21. The method of claim 20, wherein the exterior surface is folded and sealed around the interior surface using thermal bonding.

22. The method of claim 20, wherein the exterior surface is folded and sealed around the interior surface using ultrasonic bonding.

23. The method of claim 20, wherein the exterior surface is folded and sealed around the interior surface using adhesives.

24. A method of using an individually wrapped absorbent article, comprising:
unwrapping a pouch surrounding an absorbent article, the pouch formed from a unitary sheet of material comprising an interior surface and an exterior surface, at least one of the interior and exterior surfaces having a liquid absorbent capacity greater than about 10 weight percent;
separating the sheet from the absorbent article;
wiping a portion of a body surface proximate the area to which the absorbent article is to be applied with a surface of the sheet;
applying the absorbent article; and
disposing of the sheet of material.

25. The method of claim 24, further comprising removing a used absorbent article that had been previously applied, and wrapping the used absorbent article in the sheet prior to disposing of the sheet.

26. The method of claim 24, wherein the sheet further comprises at least one additive selected from the group consisting of a yeast infection treatment, a deodorant, a fragrance, a lubricant, a botanical extract, and a powder.

27. The method of claim 24, wherein the sheet is flushable.
